(19) United States
(12) Patent Application Publication
(10) Pub. No.: US 2011/0293680 A1
(43) Pub. Date: Dec. 1, 2011

(54) Composition of a Temporary Hair Thickener

(75) Inventors: Bong-Lim Jo, Gyeonggi-do (KR); Hye-Jin Moon, Gyeonggi-do (KR)

(73) Assignee: DONG SUNG PHARM. CO., LTD., SEOUL (KR)

(21) Appl. No.: 13/148,068

(22) PCT Filed: Jul. 20, 2009
(86) PCT No.: PCT/KR09/03999

§ 371 Date: Aug. 5, 2011

(30) Foreign Application Priority Data
Feb. 6, 2009 (KR) 10-2009-0009654

(51) Int. Cl. A61K 8/04; A61Q 5/00; A61K 8/97

(52) U.S. Cl. 424/401; 424/43; 424/46

(57) ABSTRACT

The present invention relates to a composition of a temporary hair thickener. In particular, the present invention relates to a composition of a temporary hair thickener comprising bamboo charcoal powder, a hydrophobic waterproof coating agent, a low grade alcohol and a propellant in a specific amount. The composition of a temporary hair thickener in accordance with the present invention can be attached to hair for a longer time as compared with the prior art hair thickeners, thereby showing improved persistency. Further, since the thickener ingredients of the composition are hardly flowed down or detached from hairs by rain or sweat, the composition of the present invention solves the problem of blotting the skin and clothes.

### TECHNICAL FIELD

The present invention relates to a composition of a temporary hair thickener comprising bamboo charcoal powder and a hydrophobic waterproof coating agent, which is characterized in that the bamboo charcoal powder adhered to the hair surface is hardly flowed down or come apart from the hairs by rain or sweat, and thus, does not contaminate the skin and clothes.

### BACKGROUND ART

Alopecia areata, also known as boldness, generally occurs due to deficiency in proteins (amino acids), minerals and vitamins to be supplied to hairs, or disorder in the function of supplying hormones and autonomic neurotransmitters promoting the growth of hairs.

In males, if testosterone, a male hormone synthesized in testicles, comes into contact with 5-alpha reductase, it is converted into dihydrotestosterone (DHT). DHT is then wrongly perceived as a normal hormone by a hormone receptor present in the hair follicle and attached thereto. Once DHT is attached to hair follicle, it prevents the assimilation of nutrients to the follicles, which in turn causes thinning out of hairs and finally shedding of hairs, thereby leading to hair loss. Therefore, an excess level of DHT in the scalp will result in baldness and this is more common in men than in women. Such DHT hair loss is then the main reason for most male hair loss. Male hair loss is medically referred as “Androgenic Alopecia areata.”

Except for genetic male pattern hair loss, all kinds of hair loss have been known as female pattern hair loss. Female pattern hair loss is ascribed to more complex reasons than those for male hair loss including nutritional deficiencies, emotional stress, environmental pollution, severe diet, intake of contaminated food with heavy metals, frequent permanents and dyeing, smoking and the like, thereby making its treatment very difficult.

Since people with alopecia areata are generally under stress, lack of self confidence thus having negative effect on personal relationship, it is preferable to treat alopecia areata in its early stage. However, even in the case of Minoxydil® known as a representative therapeutic agent for male pattern baldness and alopecia areata, its effect is largely restricted. Further, most hair tonics using various kinds of herbal extracts show little or no therapeutic effect and not medically confirmed of their effects, and there is even a risk of severe side-effects to human body. In addition, wigs having no harmful effect on humans may not be comfortable to wear and cause dermatitis seborrheica to the scalp.

In order to solve the above problems, there has been developed a temporary hair thickener and sold in commercial markets. The temporary hair thickener is designed based on the principle that a black pigment adheres to the surface of hair via electrostatic adhesion or simple physical adhesion. Based on the above property, it makes the area with baldness or thinning hair look thick and full by applying the hair thickener to the target area. Such a temporary hair thickener has several advantages that it does not form a chemical bond with the scalp or hair, there is no side-effect, and it can be easily removed by shampooing.

Conventional temporary hair thickeners usually comprise rayon fibers, carbon black or iron oxide having a fine particle size as a pigment for providing thick and full hair.

A most common composition of the temporary hair thickener has a relatively small pigment having a particle size of 40 to 150 μm (100 to 350 mesh), which can be easily adhered to the surface of hair by electrostatic force or a bonding agent when small and light pigment particles are applied thereto, thereby making the area having thin hair look as having thick hair. However, since the adhesion formed by electrostatic force is very weak and is not long-lasting, the pigment particles can be detached from the hair by a mild wind or a slight movement of the head, which may result in blotting the skin or clothes. In order to solve the above problem, there have been developed products using polyvinyl pyrrolidone, acrylate/octylacrylamaide copolymer and vinyl alcohol copolymer as an adhesive coating agent for securing strong adherence of fine pigment particles to the hair. Since the adhesive coating agent is a water-soluble polymer which can be easily formulated into a spray, it can prevent the pigment particles adhered to the hair from being blown off by wind. However, it also has disadvantage that its adhesive strength becomes weak when exposed to rain or sweat.

Further, in case of a product using rayon fiber, a regenerated fiber, there is a risk that it may be inhaled to the respiratory organ of a user or a person nearby during the spray. Therefore, although the temporary hair thickener allegedly has no negative effect on the scalp and hair, it still remains to be answered whether it would have no adverse effect on humans who are subject to inhale it.

Korean Unexamined Patent Publication No. 10-2002-0011322 discloses a hair spread composition comprising hardwood charcoal, pine needles, pine resin and/or yellow earth in a powder form. However, the hair spread composition is weakly adhered to the surface of hair and easily detached by rain or sweat, resulting in contamination of the skin and clothes. Further, it can also give users an unpleasant feeling due to the odor of pine resin.

Korean Unexamined Patent Publication Nos. 2008-0040875 and 2007-0099223 disclose shampoo and cosmetic compositions comprising charcoal powder, the objects of which are totally different from that of the present invention.

Korean Unexamined Patent Publication No. 2005-0091651 discloses a coating agent composition which comprises ferrite, silver, germanium and charcoal as additives being added to a polymer coating agent and magnetized, and used as a magnetic hair spray system for promoting hair growth. However, the above composition differs from that of the present invention in light of implementing the purposes and objectives being sought.

### DISCLOSURE

### Technical Solution

An objective of the present invention is to provide a temporary hair thickener comprising bamboo charcoal powder and acrylate copolymer as a hydrophobic waterproof coating agent, thereby minimizing toxicity to human body.

An another objective of the present invention is to solve the problem of compatibility between a hydrophobic polymer waterproof coating agent and a propellant, and provide a composition of a hair thickener in which fine pigment powder coated with the hydrophobic waterproof coating agent is highly resistant to wind, rain, sweat, and the like.

The composition of hair thickener prepared according to the present invention is characterized by comprising bamboo charcoal powder, a hydrophobic waterproof coating agent, a low grade alcohol and a propellant.

### Advantageous Effects

The hair thickener composition of the present invention comprising fine bamboo charcoal powder and a hydrophobic waterproof coating agent has a few advantages that it can minimize toxicity to human body, has excellent hair growth effect without giving any damage to the scalp and hairs, and can resolve the compatibility problem between the hydrophobic polymer coating material and a propellant. Further, the temporary hair thickener of the present invention forms a hydrophobic waterproof coating membrane, and thus provides a protective effect on the hair while preventing the hair thickener adhered to the surface of hairs from being flowed down by rain or sweat and blotting the skin and clothes.

### DESCRIPTION OF DRAWINGS

The above and other features of the present invention will now be described in detail with reference to certain exemplary embodiments thereof illustrated taken in conjunction with the accompanying drawings which are given herein below by way of illustration only, and are thus not limitative of the present invention, and wherein:

FIG. 1 shows a diagram schematically illustrating a procedure for preparing a black liquid temporary hair thickener;

FIG. 2 shows a diagram schematically illustrating a procedure for preparing a brown liquid temporary hair thickener;

FIG. 3 shows the result of hair thickening effect of a temporary hair thickener before and after the composition prepared in Example 1 is directly applied to the hair;

FIG. 4 shows the result of compatibility comparison between a propellant (dimethylether) prepared in Example 1 and a propellant (butane) prepared in Comparative Example 1;

FIG. 5 is the result of spreadability comparison between a hair thickener comprising a waterproof coating agent and a hair thickener without a waterproof coating agent; and

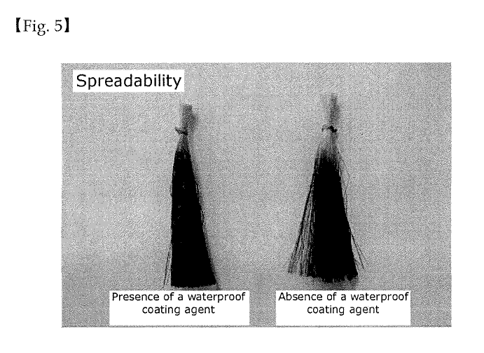

FIG. 6a shows the result of waterproof ability comparison between a hair thickener having a waterproof coating agent and a hair thickener lacking the same in water; and FIG. 6b shows the result of comparing waterproof ability between a hair thickener comprising a waterproof coating agent and a hair thickener without a waterproof coating agent in an artificial sweat solution.

### DETAILED DESCRIPTION

The hair thickener composition of the present invention comprises bamboo charcoal powder, a hydrophobic waterproof coating agent, a low grade alcohol and a propellant.

The bamboo charcoal powder is a natural substance harmless to the human body and has no side effect. Since the bamboo charcoal powder has two-fold or more of micropores as compared with hardwood charcoal powder, when applied to the hair, it circulates air flow smoothly adjacent to the scalp and hairs. Further, far infrared rays emitted therefrom can promote smooth blood circulation and metabolism of scalp capillaries and are capable of adsorbing to toxic substances such as heavy metals accumulated in the hair, thereby improving the health of hair and scalp. Such a bamboo charcoal powder is used in an amount of 1 to 60 wt %, preferably 5 to 40 wt % based on the total weight of the composition of hair thickener. If the content of the bamboo charcoal powder is less than 1 wt %, hair growth effect may not be expected due to the small amount of bamboo charcoal adhered to the hair. Meanwhile, if the content of the bamboo charcoal powder exceeds 60 wt %, the amount of a propellant is too small to maintain the propelling force and will lose its function as a propellant.

The bamboo charcoal powder used in the present invention preferably has an average particle size of 3 to 5 μm (3000 to 4000 mesh). The powder particle has a surface area of about 90 m²/g, which is relatively larger than that of the prior art rayon fiber powder having an average particle size of 40 to 150 μm (100 to 350 mesh), and thus even a small amount of the bamboo charcoal powder enables to effectively cover a desired area where hairs are scarce. Further, being a natural substance, it is not toxic to the scalp and has no side effect.

The hydrophobic waterproof coating agent is used in an amount of 1 to 40 wt %, preferably 5 to 35 wt % based on the total weight of the composition of hair thickener. If the waterproof coating agent is used less than 1 wt %, it is impossible to sufficiently apply the waterproof coating agent to the surface of hair, thereby deteriorating the waterproofing function. On the other hand, if it is used more than 40 wt %, it is not easy to spray it and apply to the hair due to the increased viscosity of a hair thickener composition. Examples of the hydrophobic waterproof coating agent suitable for the present invention include amphoteric acrylate resins that exhibit both acidic and basic properties, preferably methacryloyl ethyl betaine/acrylate copolymer (INCI Name), which is a strong hydrophobic polymer substance in which methacrylic acid is mixed with a methacryloyl ethyl betaine polymer. It can easily form a strong waterproof coating membrane when used in a formulation containing absolute alcohol and organic solvents.

A low grade alcohol is used to regulate the remaining composition of the hair thickener. It is used in an amount of 10 to 50 wt %, preferably 10 to 40 wt % based on the total amount of the composition of the hair thickener. If the low grade alcohol is used less than 10 wt %, it delays drying after it is applied to hair. Meanwhile, if is used more than 50 wt %, the composition is sprayed to a wider area than the desired hair-lacking area due to the decreased viscosity thereof, thereby blotting other areas of the skin and clothes. The low grade alcohol preferably has 1-4 carbon atoms, more preferably 2-4 carbon atoms, and includes ethanol, isopropanol, butanol, isobutanol, or a mixture thereof.

It is preferable to formulate the composition of the present invention into an aerosol spray system considering effectiveness and convenience of its use.

The propellant generally used in the aerosol spray system includes butane, iso-butane, propane, dimethylether (DME) and the like. Of them, butane, iso-butane and propane can serve as a propellant but they cannot serve as a solvent, and thus it is required to add a large amount of alcohol or an organic solvent to the composition of a temporary hair thickener. Further, they have a very low compatibility with methacryloyl ethyl betaine/acrylate copolymer used as a waterproof coating agent in the present invention, thus causing an uneven application of the waterproof coating agent to the hair when the composition is sprayed. However, dimethylether has a dual function of a propellant and a solvent, and thus it has economical advantage that it can be formulated with a small amount of alcohol or organic solvent to the composition of the present invention. Dimethylether is preferably used in this invention as it is. In particular, since dimethylether has high compatibility with methacryloyl ethyl betaine/acrylates copolymer used as a waterproof coating agent of the present invention, it can mediate the waterproof coating agent to be evenly attached to hair. Dimethylether is used in an amount of 25 to 60 wt %, preferably 35 to 55 wt % based on the total amount of the composition of hair thickener. If the propellant is used less than 25 wt %, its propelling pressure becomes too low to spray the composition evenly. Meanwhile, if it exceeds 60 wt %, the propelling pressure becomes excessive, and thus the excessive high propelling pressure may dishevel a user's hairstyle.

The bamboo charcoal used in the present invention is black. Thus, in order to create a natural dark brown color, an organic pigment may be used in an amount of 0.1 to 10% by weight based on the total weight of the composition of a hair thickener. The organic pigment can be used in the preparation of a hair thickener composition having various colors similar to the human hairs by varying the mixed ratio of the organic pigment. The organic pigment is used as a temporary hair color, which dyes one's hair via simple physical adhesion, unlike the temporary hair coloring composition. Namely, the organic pigment which is insoluble to water or a conventional solvent is adhered to the surface of hair by electrostatic force or an adhesive. Examples of such a temporary hair color widely used in the art include a hair mascara for covering grey hair, a color spray, a color mousse and the like. The organic pigment used in the present invention can be prepared by adding a metal salt to a water-insoluble dye or water-soluble dye followed by precipitation. It has characteristics of vivid color, strong coloring strength and various kinds of colors. Therefore, it has been widely used in printing ink, temporary hair color and the like. Such an organic pigment includes one or more azo groups (—N═N—) within its molecule, and is preferably selected from the group consisting of Pigment Red 202 (CI 15850), Pigment Red 205 (CI 15630), Pigment Red 206 (CI 15630), Pigment Red 207 (CI 15630), Pigment Red 208 (CI 15630), Pigment Red 219 (CI 15800), Pigment Red 220 (CI 15880), Pigment Red 221 (CI 12120), Pigment Red 228 (CI 12085), Pigment Red 404 (CI 12315), Pigment Orange 203 (CI 12075), Pigment Orange 204 (CI 21110), Pigment Orange 401 (CI 11725), Pigment Orange 205 (CI 21090), Pigment Yellow 401 (CI 11680), Pigment Blue 404 (CI 74160) and mixtures thereof. Representative examples thereof are illustrated by the following formulae 1 to 3:

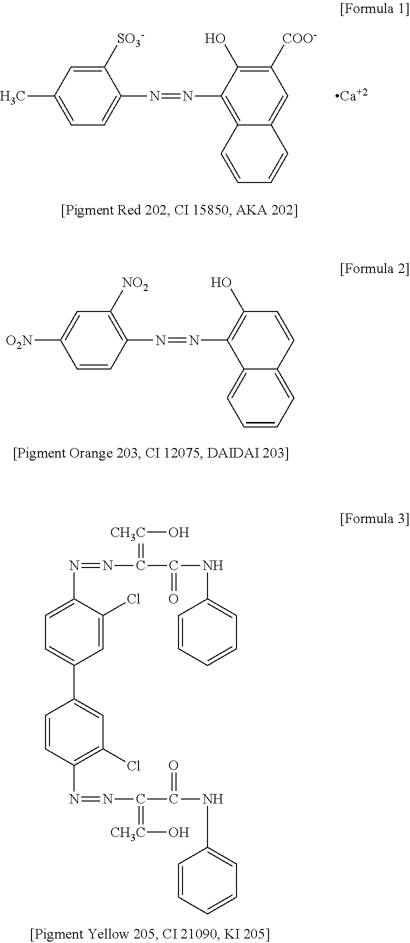

The composition of a temporary hair thickener comprising the ingredients in accordance with the present invention can fill the area where hair fall out, and the hydrophobic waterproof coating agent can form a strong waterproof coating membrane for preventing the ingredients applied onto the hair from separating. Through the effective application and strong waterproofing coating, the bamboo charcoal and organic pigment adhered to the surface of hairs are highly resistant to flow down or detach from hairs by rain or sweat, and thus, do not contaminate the skin and clothes.

### EXAMPLES

The following examples illustrate the invention and are not intended to limit the same.

### Example 1

### Preparation of a Black Composition of Hair Thickener

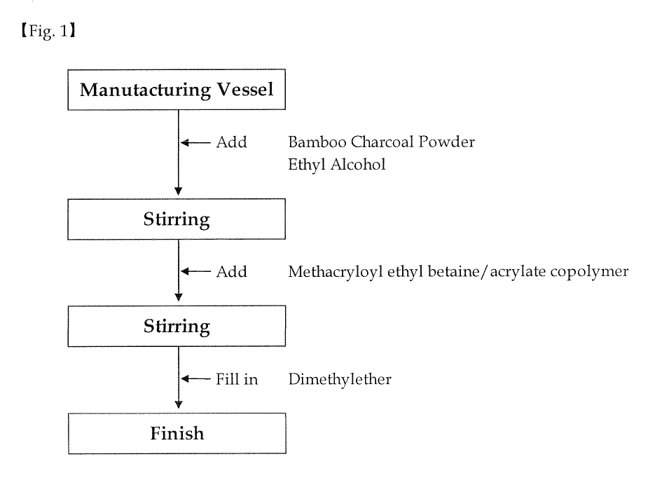

Referring to the procedure illustrated in FIG. 1, a hydrophobic waterproof coating agent, bamboo charcoal powder and a low grade alcohol are mixed in order with stirring according to the amount (wt %) as described in Table 1, and a propellant was filled thereto, to thereby obtain a black composition of hair thickener.

### Example 1

### Preparation of a Brown Composition of Hair Thickener

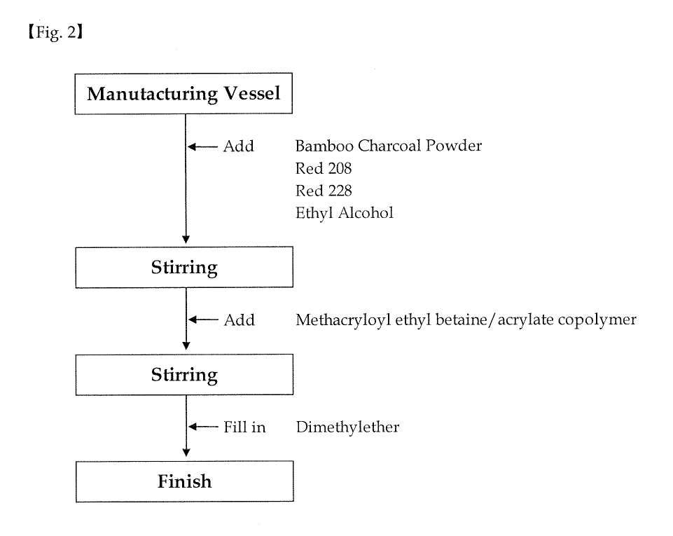

Referring to the procedure illustrated in FIG. 2, a hydrophobic waterproof coating agent, bamboo charcoal powder, a low grade alcohol and an organic pigment are mixed in order with stirring according to the amount (wt %) as described in Table 1, and a propellant was filled thereto, to thereby obtain a brown composition of hair thickener.

### Comparative Example 1

### Preparation of a Black Composition of Hair Thickener

The black composition of hair thickener was prepared according to the same method as described in Example 1.

### Comparative Example 2

### Preparation of a Black Composition of Hair Thickener

The black composition of hair thickener was prepared according to the same method as described in Example 1, except that a water-soluble coating agent widely used in the preparation of the prior art temporary hair thickener is used instead of the hydrophobic waterproof coating agent.

TABLE 1

| | | | | Comp. Ex. | Comp. Ex. |
|---|---|---|---|---|---|
| | | Ex. 1 | Ex. 2 | 1 | 2 |
| Bamboo charcoal | | 15 | 12 | 15 | 15 |
| powder | | | | | |
| Hydrophobic waterproof | | 22 | 22 | 22 | — |
| coating agent¹⁾ | | | | | |
| Water-soluble coating | | | | — | 22 |
| agent²⁾ | | | | | |
| Lower alcohol³⁾ | | 18 | 20.56 | 18 | 18 |
| Organic | Red 202 | — | 0.18 | — | — |
| pigment⁴⁾ | Red 228 | — | 0.26 | — | — |
| Propellant | Dimethyl | 45 | 45 | — | 45 |
| | ether | | | | |
| | butane | — | — | 45 | — |
| Total | | 100 | 100 | 100 | 100 |

¹⁾Hydrophobic waterproof coating agent: methacryloyl ethyl betaine/acrylate copolymer
²⁾Water-soluble coating agent: polyvinyl pyrrolidone
³⁾Low grade alcohol: ethyl alcohol
⁴⁾Organic pigment: Red 202 (CI 15850), Red 228 (CI 12085)

### Test Example 1

### Effect of Application on Hair

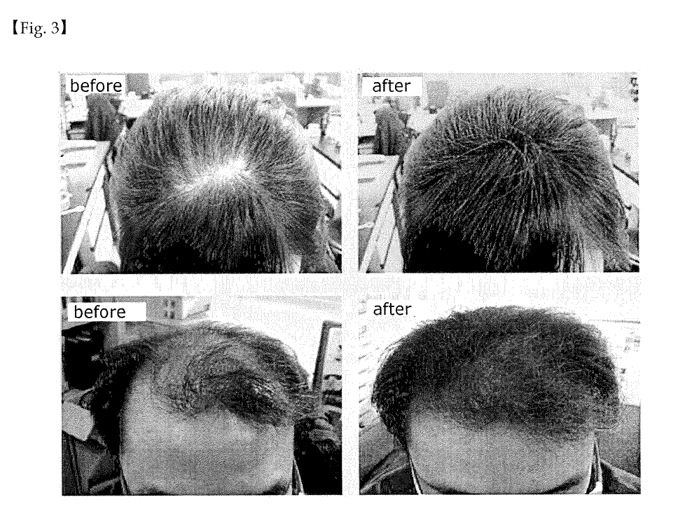

In order to investigate the effectiveness of the composition of a temporary hair thickener in accordance with the present invention, the composition of Example 1 was applied to the bald spots of a person's head, and it was monitored whether the area becomes rich in hair. The result is shown in FIG. 3.

### Test Example 2

### Comparison of Comparability between a Hydrophobic Waterproof Coating Agent and a Propellant

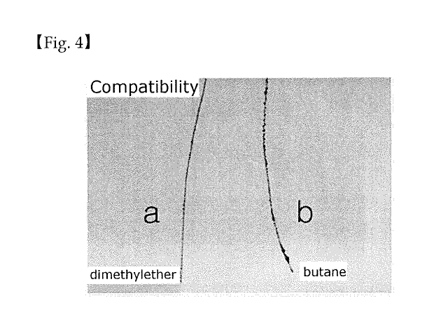

In order to compare comparability between the hydrophobic waterproof coating agent and the propellant in the compositions of Example 1 and Comparative Example 1, respectively, the surface of hair to which the composition was applied was observed. The result is shown in FIG. 4.

### Test Example 3

### Confirmation on the Formation of a Hydrophobic Waterproof Coating Membrane

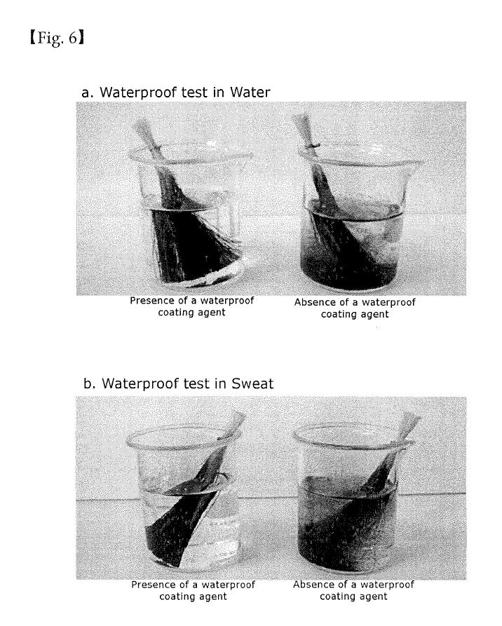

In order to examine the capability of the composition of hair thickener to form a hydrophobic waterproof coating membrane, after each of the compositions prepared in Example 1 and Comparative Example 1 was applied to an artificial white hair, the resulting hair was dried at 20□ for a minute and soaked in pure water and an artificial sweat solution, respectively. At this time, whether the bamboo charcoal and organic pigment applied to the hair are dissolved in the solution was observed, and the result is shown in FIGS. 6a and 6b. Here, the artificial sweat solution was prepared by mixing 1.0 wt % of sodium chloride, 0.1 wt % of lactic acid, 0.1 wt % of disodium hydrogen orthophosphate, 0.02 wt % of histidine hydrochloride and 98.78 wt % of distilled water.

According to the results of Test Example 1, as illustrated in FIG. 3, the composition of a temporary hair thickener in accordance with the present invention was effectively applied to the crown of the head being scarce of hair, and thus, made the area look like enriched with hair.

According to the result of Test Example 2, in case of the composition of a temporary hair thickener (Example 1) using dimethylether as a propellant, the composition was uniformly and smoothly applied to the surface of hair (FIG. 4a). However, in case of the composition using butane as a propellant (Comparative Example 1), it was not uniformly applied and formed into aggregates onto the surface of hair (FIG. 4b).

According to the result of Test Example 3, when the artificial white hair to which the composition of a temporary hair thickener comprising the hydrophobic waterproof coating agent (Example 1) was applied was soaked in pure water and the artificial sweat solution, respectively, as shown in FIGS. 6a and 6b, the thickener ingredients applied thereto were not dissolved into the solution. On the other hand, when the artificial white hair to which the composition having no hydrophobic waterproof coating agent (Comparative Example 1) was applied was soaked in pure water and the artificial sweat solution, respectively, as shown in FIGS. 6a and 6b, the thickener ingredients were dissolved into the solution, which makes the solution cloudy with the dissolved charcoal powder.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

1. A composition of a temporary hair thickener comprising bamboo charcoal powder, a hydrophobic waterproof coating agent, a low grade alcohol and a propellant.

2. The composition of a temporary hair thickener according to claim 1, which further comprises an organic pigment.

3. The composition of a temporary hair thickener according to claim 1, wherein the bamboo charcoal powder is used in an amount of 1 to 60 wt % based on the total amount of the composition of a temporary hair thickener.

4. The composition of a temporary hair thickener according to claim 1, wherein the hydrophobic waterproof coating agent is used in an amount of 1 to 40 wt % based on the total amount of the composition of a temporary hair thickener.

5. The composition of a temporary hair thickener according to claim 1, wherein the low grade alcohol is used in an amount of 10 to 50 wt % based on the total amount of the composition of a temporary hair thickener.

6. The composition of a temporary hair thickener according to claim 1, wherein the propellant is used in an amount of 25 to 60 wt % based on the total amount of the composition of a temporary hair thickener.

7. The composition of a temporary hair thickener according to claim 2, wherein the organic pigment is used in an amount of 0.1 to 10 wt % based on the total amount of the composition of a temporary hair thickener.

8. The composition of a temporary hair thickener according to claim 1, wherein the bamboo charcoal powder has an average particle size of 3 to 5 μm.

9. The composition of a temporary hair thickener according to claim 1, wherein the hydrophobic waterproof coating agent is methacryloyl ethyl betaine/acrylate copolymer.

10. The composition of a temporary hair thickener according to claim 1, wherein the low grade alcohol is an alcohol having 1-4 carbon atoms.

11. The composition of a temporary hair thickener according to claim 1, wherein the propellant is dimethylether.

12. The composition of a temporary hair thickener according to claim 2, wherein the organic pigment is one or more selected from the group consisting of Pigment Red, Pigment Orange, Pigment Yellow and Pigment Blue.

13. The composition of a temporary hair thickener according to claim 3, wherein the bamboo charcoal powder has an average particle size of 3 to 5 μm.

14. The composition of a temporary hair thickener according to claim 4, wherein the hydrophobic waterproof coating agent is methacryloyl ethyl betaine/acrylate copolymer.

15. The composition of a temporary hair thickener according to claim 5, wherein the low grade alcohol is an alcohol having 1-4 carbon atoms.

16. The composition of a temporary hair thickener according to claim 6, wherein the propellant is dimethylether.

17. The composition of a temporary hair thickener according to claim 7, wherein the organic pigment is one or more selected from the group consisting of Pigment Red, Pigment Orange, Pigment Yellow and Pigment Blue.
